(19) United States
(12) Patent Application Publication
(10) Pub. No.: US 2011/0282307 A1
(43) Pub. Date: Nov. 17, 2011

(54) Leak Proof Mucus Extractor

(76) Inventor: Mei-Sheng TENG, Kowloon (CN)

(21) Appl. No.: 12/779,685

(22) Filed: May 13, 2010

(51) Int. Cl. A61M 1/00

(52) U.S. Cl. 604/319

(57) ABSTRACT

A leak proof mucus extractor in accordance with the present invention comprises: an inner suction tube, an outer tube, a tube seat, a connecting handle, a torsion spring and a bracket; it is characterized in that: the outer tube has a valve at a front end thereof and is engaged on the inner suction tube which has a suction hole, the outer tube is fixed to the tube seat, and the inner suction tube is fixed to the bracket which is formed with a suction-pump-coupling port, the connecting handle has an upper end hinged to the tube seat and a lower end hinged to the bracket and the torsion spring in such a manner that both ends of the torsion spring are tensioned between the connecting handle and the bracket.

### BACKGROUND OF THE INVENTION

1. Field of the Invention

The present invention relates to a leak proof mucus extractor, and more particularly to a leak proof mucus extractor which is used in medical facilities and homes to help patients clear respiratory tract obstructions.

2. Description of the Prior Art

Mucus extractor works in such a way that the suction tube is inserted in the patient's respiratory tract and the suction pump is turned on to produce a negative pressure in the suction tube, mucus is sucked into the suction tube from the apertures in the distal end thereof, and then the respiratory tract is cleaned by pulling out the suction tube.

In real application, the operator may encounter the following problems: the negative pressure in the suction tube is low, or the negative pressure may suddenly disappear because of an operation mistake. Therefore, the mucus which has been sucked into the suction tube falls back into the respiratory tract by gravity, causing new respiratory tract obstruction or the mucus is sucked into the patient's lung, as a result, it may fail to clean the respiratory tract, or even make the patient worse. If the mucus flows or leaks out of the suction tube after being sucked thereinto, it will cause environmental contamination, and for a medial location with extremely high hygiene requirements, this is in no way different from a disaster.

The present invention has arisen to mitigate and/or obviate the afore-described disadvantages.

### SUMMARY OF THE INVENTION

The primary object of the present invention is to provide a simply structured and easy-to-use leak proof mucus extractor.

To achieve the above object, a leak proof mucus extractor in accordance with the present invention comprises: an inner suction tube, an outer tube, a tube seat, a connecting handle, a torsion spring and a bracket; it is characterized in that: the outer tube has a valve at a front end thereof and is engaged on the inner suction tube which has a suction hole, the outer tube is fixed to the tube seat, and the inner suction tube is fixed to the bracket which is formed with a suction-pump-coupling port, the connecting handle has an upper end hinged to the tube seat and a lower end hinged to the bracket and the torsion spring in such a manner that both ends of the torsion spring are tensioned between the connecting handle and the bracket.

When in use, the mucus extractor is inserted into a patient's respiratory tract and the user presses the connecting handle and turns on the suction pump, pushing the outer tube move back, so that the front end of the inner suction tube pushes open the valve at the front end of the outer tube, and the mucus is sucked under the negative pressure from the respiratory tract into the suction hole at the front end of the inner suction tube. The user can stop the mucus sucking operation at any time as he wants to simply by releasing the connecting handle. The torsion spring will push the outer tube forward to move back to its original position, and the valve at the front end of the outer tube will automatically be closed, plus with the effect of the negative pressure, the outer tube can firmly encloses the inner suction tube, preventing the occurrence of mucus leakage or backflow.

### BRIEF DESCRIPTION OF THE DRAWINGS

FIG. 1 is an exploded view of a leak proof mucus extractor in accordance with the present invention;

FIG. 2 is a front view in accordance with the present invention, showing that the leak proof mucus extractor is in a sucking state;

FIG. 3 is a cross sectional view in accordance with the present invention, showing that the leak proof mucus extractor is in a sucking state;

FIG. 4 is a side view in accordance with the present invention, showing that the leak proof mucus extractor is in a sucking state;

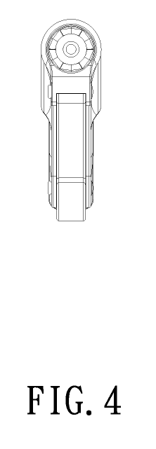

FIG. 5 is a front view in accordance with the present invention, showing that the leak proof mucus extractor is in a closed state;

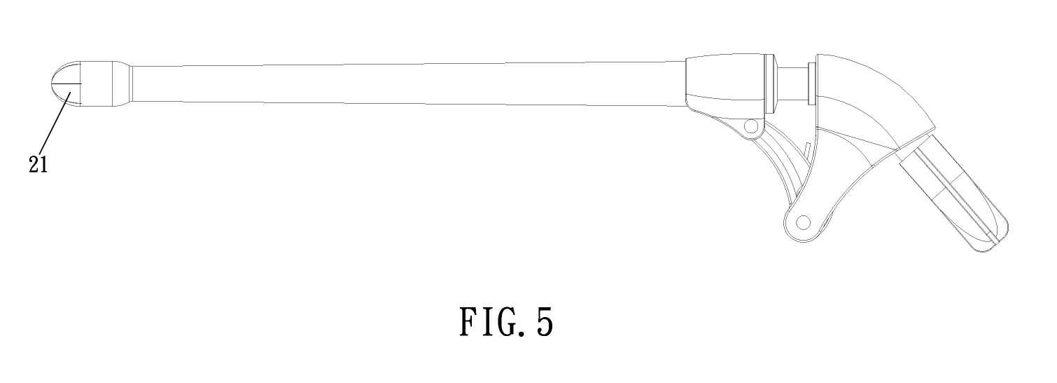

FIG. 6 is a cross sectional view in accordance with the present invention, showing that the leak proof mucus extractor is in a closed state; and

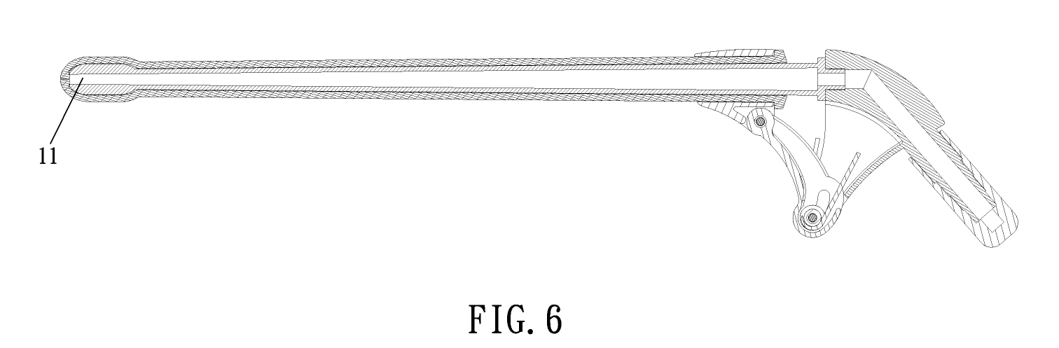

FIG. 7 is a side view in accordance with the present invention, showing that the leak proof mucus extractor is in a closed state.

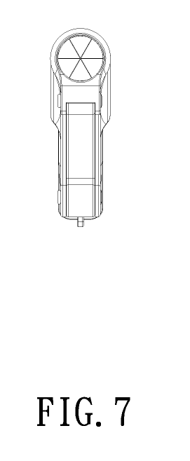

### DETAILED DESCRIPTION OF THE PREFERRED EMBODIMENT

The present invention will be clearer from the following description when viewed together with the accompanying drawings, which show, for purpose of illustrations only, the preferred embodiment in accordance with the present invention.

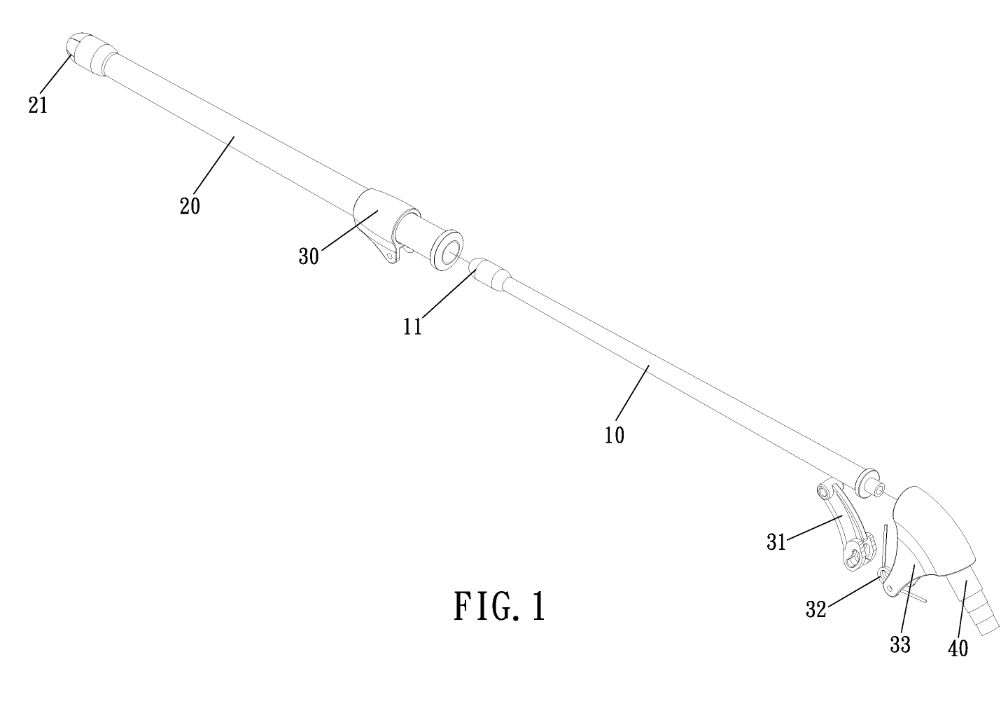

Referring to FIG. 1, a leak proof mucus extractor in accordance with the present invention comprises an inner suction tube 10, an outer tube 20, a tube seat 30, a connecting handle 31, a torsion spring 32 and a bracket 33. The present invention is characterized in that: the outer tube 20 has a valve 21 at the front end thereof and is engaged on the inner suction tube 10 which has a suction hole 11. The outer tube 20 is fixed to the tube seat 30, and the inner suction tube 10 is fixed to the bracket 33 which is formed with a suction-pump-coupling port 40. The connecting handle 31 has an upper end hinged to the tube seat 30 and a lower end hinged to the bracket 33 with the suction-pump-coupling port 40, and the torsion spring 32 in such a manner that both ends of the torsion spring 32 are tensioned between the connecting handle 31 and the bracket 33. The present invention is simply structured and easy to operate.

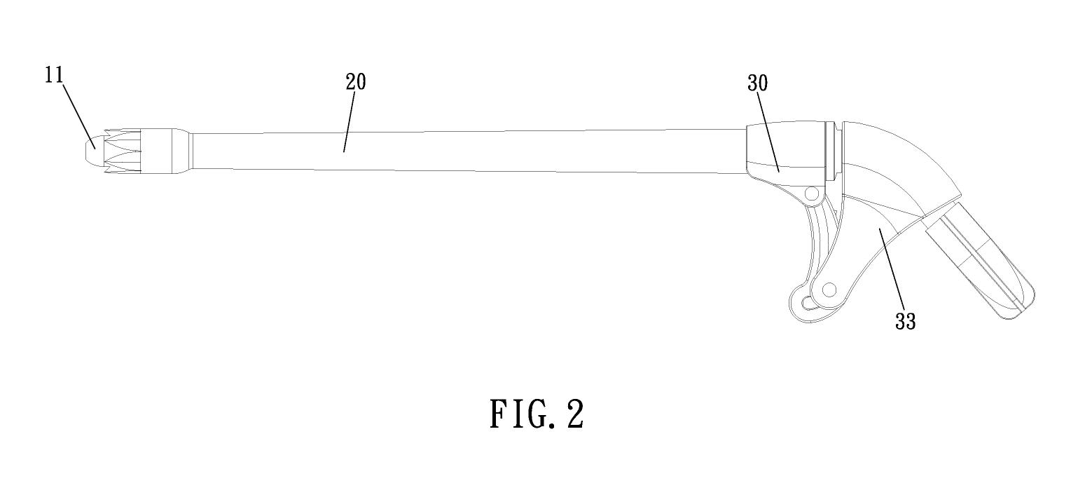

Referring to FIG. 2, when in use, the mucus extractor in accordance with the present invention is inserted into a patient's respiratory tract and the suction-pump-coupling port 40 is connected to a suction pump which is set on a turned on state, the user presses the connecting handle 31, and the outer tube 20 is driven by the connecting handle 31 to move back and forth along the inner suction tube 10, so that the front end of the inner suction tube 10 will push open the valve 21 at the front end of the outer tube 20 automatically, and the mucus will be sucked under the negative pressure from the respiratory tract into the suction hole 11 at the front end of the inner suction tube 10.

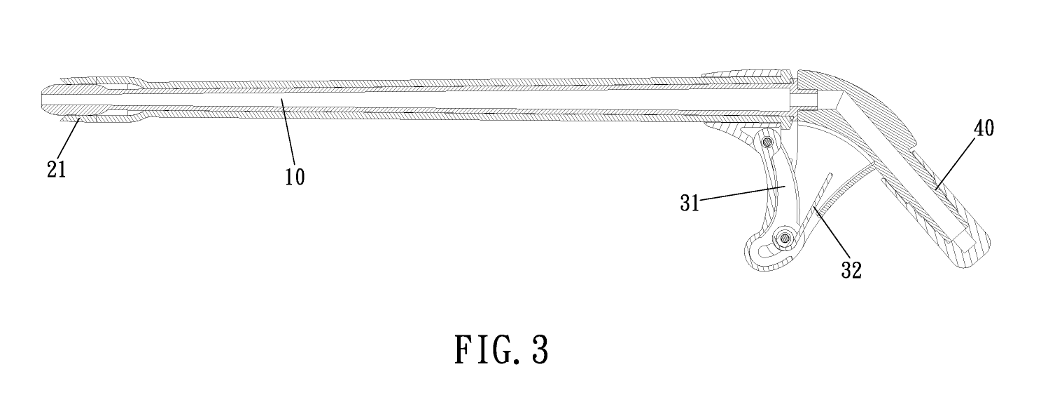

Referring to FIG. 3, when mucus suction is done, the user releases the connecting handle 31, the torsion spring 32 will make the connecting handle 31 rotate, and then the connecting handle 31 will push the tube seat 30 and the outer tube 20 to move forward along the inner suction tube 10. When the front end of the outer tube 20 moves over (to a position ahead of) the front end of the inner suction tube 10, the valve 21 at the front end of the outer tube 20 will automatically be closed, plus with the effect of the negative pressure, the outer tube 20 can firmly encloses the inner suction tube 10, preventing the occurrence of mucus leakage or backflow.

While we have shown and described various embodiments in accordance with the present invention, it is clear to those skilled in the art that further embodiments may be made without departing from the scope of the present invention.

What is claimed is:

1. A leak proof mucus extractor comprising: an inner suction tube, an outer tube, a tube seat, a connecting handle, a torsion spring and a bracket; characterized in that: the outer tube has a valve at a front end thereof and is engaged on the inner suction tube which has a suction hole, the outer tube is fixed to the tube seat, and the inner suction tube is fixed to the bracket which is formed with a suction-pump-coupling port, the connecting handle has an upper end hinged to the tube seat and a lower end hinged to the bracket and the torsion spring in such a manner that both ends of the torsion spring are tensioned between the connecting handle and the bracket.
